(19) United States
(12) Patent Application Publication
(10) Pub. No.: US 2014/0048061 A1
(43) Pub. Date: Feb. 20, 2014

(54) SYSTEM AND METHOD FOR ADMINISTERING ANESTHETICS WHILE PERFORMING CPR

(71) Applicants: Demetris Yannopoulos, Edina, MN (US); Keith G. Lurie, Minneapolis, MN (US)

(72) Inventors: Demetris Yannopoulos, Edina, MN (US); Keith G. Lurie, Minneapolis, MN (US)

(21) Appl. No.: 13/964,882

(22) Filed: Aug. 12, 2013

Related U.S. Application Data
(60) Provisional application No. 61/682,117, filed on Aug. 10, 2012.

(51) Int. Cl. A61H 31/00; A61M 5/00; A61N 1/39; A61M 16/01; A61M 11/00

(52) U.S. Cl. CPC A61H 31/00; A61M 16/01; A61M 11/00; A61N 1/3987; A61M 5/00
USPC 128/200.14; 128/203.12

(57) ABSTRACT

According to one embodiment, a device for delivering an anesthetic to an individual to reduce reperfusion injury during the performance of cardiopulmonary resuscitation (CPR) is provided. The device includes a patient connection mechanism for coupling with an airway of the individual and an anesthetic delivery mechanism for receiving the anesthetic and for delivering the anesthetic to the individual via the patient connection mechanism. In some embodiments, the anesthetic is delivered with the assistance of an intrathoracic pressure regulation (IPR) mechanism or an impedance threshold device (ITD) coupled with the patient connection mechanism. The IPR mechanism may be configured to change a pressure in the airway and a thorax of the individual via application of a vacuum source. The ITD device may be configured to prevent respiratory gases from entering the lungs for at least some time during a decompression or relaxation phase of CPR.

### CROSS-REFERENCES TO RELATED APPLICATIONS

This application claims priority to Provisional U.S. Patent Application No. 61/682,117 filed Aug. 10, 2012, entitled “System and Method for Administering Anesthetics While Performing CPR,” the entire disclosure of which is hereby incorporated by reference, for all purposes, as if fully set forth herein.

The application is related to the following U.S. patents applications, each of which are incorporated by reference herein: Ser. Nos. 07/686,542; 08/058,195; 08/226,431; 07/977,498; 08/149,204; 08/403,009; 08/747,371; 08/950,702; 09/019,843; 09/095,916; 09/168,049; 09/197,286; 09/315,396; 09/386,868; 09/614,064; 09/532,601; 09/533,880; 09/564,889; 09/546,252; 09/704,231; 09/854,404; 09/854,238; 09/966,945; 09/967,029; 10/119,203; 10/158,528; 10/251,080; 10/224,263; 10/255,319; 10/401,493; 10/410,229; 10/396,007; 10/426,161; 10/460,558; 10/660,366; 10/660,462; 10/765,318; 10/796,875; 10/920,678; 11/034,996; 11/051,345; 11/127,993; 11/735,924; 11/679,693; 11/690,065; 11/735,320; 60/912,891; 60/917,602; 60/944,735; 60/947,346; 11/871,879; 11/862,099; 11/949,490; 12/119,374; 12/141,831; 12/141,864; 12/165,366; 12/843,512; 61/304,148; 61/218,763; 12/723,205; 12/819,959; 61/368,150; 13/026,459; 13/189,330; 13/226,211; 61/606,153; 11/127,055; 11/526,956; 61/291,211; 61/296,391; 12/792,333; 12/793,374; 61/361,208; 61/485,944; 13/175,670; 61/509,994; and 61/577,565.

### BACKGROUND

The study of the physiology effects of cardiac arrest has been a particular area of interest in recent decades. This is due mainly to cardiac arrest remaining the leading cause of death in the United States. As a result of this focus, a number of approaches to treating cardiac arrest have been developed, which have resulted in significant clinical advances in the field. Despite this progress, greater than 80% of patients who experience sudden and unexpected out of hospital cardiac arrest (OHCA) cannot be successfully resuscitated. The prognosis is particularly grim in patients with a prolonged time between cardiac arrest and the start of cardiopulmonary resuscitation (CPR). A common, harmful, and previously under-recognized contributor to patient morbidity and mortality is reperfusion injury (RI), which typically occurs or is induced after a prolonged period of no blood flow in the OHCA setting.

These persistently high mortality rates have encouraged exploration of new approaches to cardiac arrest with a goal of improving patient outcomes. Despite intensive research over half a century, only small improvements in resuscitation survival outcomes have been observed. Most efforts in the field have focused on improving hemodynamics during CPR. Ideally, any new approaches would complement previously determined methods that improve circulation to the heart and brain after cardiac arrest.

### BRIEF SUMMARY OF THE INVENTION

The embodiments described herein provide devices, systems, and methods for reducing reperfusion injury after blood flow is reinitiated following ischemia. According to one embodiment, a device for delivering an anesthetic to an individual to reduce reperfusion injury during the performance of cardiopulmonary resuscitation (CPR) is provided. The device includes a patient connection mechanism for coupling with an airway of the individual. The device also includes a pressure regulation device that is configured to regulate a negative intrathoracic pressure of the individual while CPR is being performed on the individual. In some embodiments, the pressure regulation device is an intrathoracic pressure regulation (IPR) mechanism that is coupled with the patient connection mechanism. The IPR mechanism may be configured to change a pressure in the airway and a thorax of the individual via the application of a vacuum source such as by reducing the pressure in the thorax below atmospheric pressure in order to enhance the circulation of blood in the body. In other embodiments, the pressure regulation device may be an impedance threshold device (ITD) that is coupled with the patient connection mechanism and that prevents respiratory gases from entering the lungs for at least some time during the decompression or relaxation phase of CPR. The device may further include an anesthetic delivery mechanism for receiving the anesthetic and for delivering the anesthetic to the individual in cardiac arrest undergoing CPR via the patient connection mechanism. Delivery of the anesthetic throughout the individual's body is enhanced by the use of the pressure regulation device (i.e., IPR and/or ITD).

In some embodiments, the device may include a housing and the anesthetic delivery mechanism may include a chamber for receiving a vial of the anesthetic. In some embodiments, the housing may have an upper chamber and a lower chamber separated by a filter. The lower chamber may include an impedance threshold device (ITD) and the upper chamber may include an inlet port for receiving a gas to be delivered to the individual via the patient connection mechanism. The ITD prevents the flow of respiratory gases to the lungs for at least some time during the decompression or relaxation phase of CPR in order to enhance circulation in the individual, thereby facilitating the delivery of the anesthetic to the body. In some embodiments, the upper chamber may also include an absorbent buffer. The device may additionally include an aerosolizer that is configured to aerosolize the anesthetic.

According to another embodiment, a method of performing cardiopulmonary resuscitation is provided. The method includes repeatedly compressing an individual's chest and administering an anesthetic to the individual receiving cardiopulmonary resuscitation. Repeatedly compressing the individual's chest includes compressing the chest during a compression phase followed by a decompression or relaxation phase. The anesthetic may be administered through the lungs of the individual within 3 minutes of, or prior to, initiating cardiopulmonary resuscitation. In some embodiments, the anesthetic may be administered through the lungs of the individual within 30 seconds of initiating cardiopulmonary resuscitation and/or administered prior to compressing the individual's chest or applying a defibrillating shock.

In some embodiments, the anesthetic may be administered while performing an enhanced circulation cardiopulmonary resuscitation procedure. The enhanced circulation cardiopulmonary resuscitation procedure may including: a procedure involving an impedance threshold device (ITD) where respiratory gases are prevented from entering the lungs for at least some time during the decompression or relaxation phase, manual or automated active compression decompression (ACD) CPR where the individual's chest is actively lifted during the decompression phase, a procedure involving an intrathoracic pressure regulator (IPR) device where the IPR mechanism changes a pressure in the airway and a thorax of the individual via application of a vacuum source, or any combination thereof. In any event, the anesthetic may be administered prior to restoration of a heartbeat and/or may modulate the autonomic nervous system.

In some embodiments, the anesthetic may include: isoflurane, sevoflurance, xenon, helium, desflurane, enflurance, halothane, methoxyflurance, nitrous oxide, propofol, ketamine, etomidate, amobarbital, methohexital, thiopental, a barbiturate, a benzodiazepine, and the like. In some embodiments, the anesthetic may be a volatile anesthetic. In some embodiments, two or more doses of the anesthetic or two or more anesthetics may be administered to the individual receiving cardiopulmonary resuscitation. In such embodiments, the two or more anesthetics may be administered substantially simultaneously.

The anesthetic may be administered to the individual via inhalation, intravenously, intramuscularly, intraosseously, and the like. The anesthetic may be administered to the individual for at least 30 seconds, and more commonly may be administered to the individual for at least 3-5 minutes.

According to another embodiment, a method of reducing reperfusion injury after a period of ischemia is provided. The method includes administering an anesthetic to an individual prior to or approximate the time blood supply is returned to tissue of the individual. The anesthetic may be administered during cardiopulmonary resuscitation.

According to another embodiment, a method may include performing cardiopulmonary resuscitation to an individual by repeatedly compressing an individual's chest where the chest is compressed during a compression phase followed by a decompression or relaxation phase. The method may also include administering anesthesia during the performance of cardiopulmonary resuscitation to modulate the autonomic nervous system of the individual.

### BRIEF DESCRIPTION OF THE DRAWINGS

The present invention is described in conjunction with the appended figures:

FIG. 1 illustrates a device for administering an anesthetic to an individual according to one embodiment of the invention.

FIG. 2 illustrates another device for administering an anesthetic to an individual according to one embodiment of the invention.

FIGS. 3-5 illustrate various methods according to embodiments of the invention.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

Embodiments of the invention provide devices, systems, and methods for reducing reperfusion injury after blood flow is reinitiated during CPR to treat cardiac arrest. During cardiac arrest, the absence of oxygen and/or other nutrients can create conditions that result in inflammation and/or other damage to tissue and/or cell components. This occurs when the heart stops beating during a cardiac arrest. Blood supply or flow is interrupted to an individual's heart and brain, which may cause damage to the heart and brain tissue, or even death if left untreated for an extended period of time. Cardiopulmonary resuscitation (CPR) may be performed in order to restart beating of the heart or to reestablish circulation of blood within the body. CPR typically involves performing chest compressions, either manually or with the assistance of a machine or device; performing artificial respiration to provide oxygen to the body, either manually (e.g., mouth to mouth) or with the assistance of a machine or device; or some combination thereof.

The embodiments described herein focus on an approach to restoring brain and heart function after cardiac arrest by utilizing inhalational anesthetic agents, or other anesthetic agents, to reduce or prevent reperfusion injury. It is believed that survival with favorable neurological outcomes can be significantly improved by preventing the molecular, cellular, and metabolic changes that result from abrupt reperfusion, especially after prolonged untreated cardiac arrest (i.e., reintroduction of blood flow during the initiation of CPR). The significant improvement may be due to reperfusion injury being of greater consequence than the injury caused by the duration of initial ischemia itself.

Anesthetic agents have been administered to patients to protect against reperfusion injury protection. It is commonly believed, however, that patients suffering from cardiac arrest will not significantly benefit from the administration of such drugs. In contrast to this conventional thought, Applicants have discovered that administration of anesthetic agents and/or other volatile gases, especially prior to or during the initiation of CPR, significantly protects the patient and/or enhances the effectiveness of CPR. The effectiveness of the anesthetic agents and/or other volatile gases is further enhanced by the use of the enhanced circulation procedures described herein (i.e., ACD CPR, ACD CPR with an IPR or ITD device, and the like). The increased circulation afforded by these procedures results in a substantially more rapid delivery and distribution of the anesthetic agents and/or other volatile gases, which delivery and distribution may not be as quick and/or effective with the use of conventional CPR procedures. Stated differently, the enhanced circulation procedures more effectively draw blood back into the chest and lungs where it will be introduced to the administered anesthetic. As such, when the chest is subsequently compressed during CPR, the blood, which now includes the anesthetic, will be carried throughout the body and into the body's extremities. In this manner, the delivery and distribution of the anesthetic may be enhanced via the enhanced CPR methods described herein.

Conceptually, the time window after unexpected out of hospital cardiac arrest (OHCA) can be divided into 3 phases: 1) the electrical phase (i.e., minutes 0-4 after collapse) where defibrillation is most commonly successful, 2) the circulatory phase (i.e., between 4 minutes and 8 minutes) where improved flow is generally needed to enhance survival, and 3) the metabolic phase (i.e., greater than 8 minutes) where conventional procedures offer patients little hope of survival. Ideally, patients who suffer from OHCA would receive bystander CPR and rapid defibrillation soon after OHCA. Unfortunately, in most emergencies, only a small percentage of patients receive CPR before the arrival of first responders and only 30% are found in ventricular fibrillation (VF), which commonly translates into an absence of circulation for an average of 8-10 min, placing the patient in or near the metabolic phase.

It is believed that after untreated arrest, reintroduction of blood flow during the metabolic phase contributes significantly to the overall tissue damage. As such, reduction and/or prevention of cellular injury associated with reperfusion should be a therapeutic target at the start of CPR in the vast majority of patients. The embodiments described herein focuses on a readily adoptable protective strategy to limit critical organ reperfusion injury that occurs at the initiation of CPR.

Because of the limited window of opportunity to treat reperfusion injury (e.g., 2-3 min from the re-introduction of blood flow) and the complexity of treating patients with OHCA, treatment strategies to reduce or prevent reperfusion injury ideally would have the following characteristics: 1) be available for application within the first 1-2 min of CPR, 2) be non-invasive, 3) be simple to implement, and 4) cause no delay in the initiation of chest compressions. The embodiments described herein offer a simple approach that utilizes chest compressions and airway access to the lungs so that professional basic life support (BLS) providers can provide treatment in the field under a wide variety of conditions.

Reperfusion injury occurs very early during reintroduction of blood flow during CPR. It is believed that after prolonged untreated arrest, reintroduction of blood flow contributes significantly to the overall injury (reperfusion injury). In prior applications, Applicants have described protective strategies that reduce or prevent reperfusion injury and improve neurologically sound survival after 15 minutes of untreated cardiac arrest. These observations have been have incorporated into U.S. patent application Ser. No. 13/554,986 filed on Jul. 20, 2012, titled “ENHANCED GUIDED ACTIVE COMPRESSION DECOMPRESSION CARDIOPULMONARY RESUSCITATION SYSTEMS AND METHODS”, and US patent application Ser. No. 13/554,458 filed on Jul. 20, 2012, titled “METHODS AND SYSTEMS FOR REPERFUSION INJURY PROTECTION AFTER CARDIAC ARREST”, the entire disclosures of which are incorporated herein by reference. Pharmacological agents (e.g. cyclosporine A) may provide similar injury protection results.

The embodiments described herein further limit or reduce reperfusion injury through the administration of an anesthetic during the initiation of CPR. It is believed that the administration of the anesthetic drastically alters the biological milieu, thus protecting vital organs from injury and thereby leading to better outcomes even after prolonged untreated cardiac arrest. For example, conventional belief suggests that brain death is essentially imminent after 4-6 minutes of ischemia. Preliminary results utilizing the improvements in resuscitation strategies described herein, however, suggest that good neurologic function can be attained even after 15 minutes of ischemia. As such, the potential for redefining heart and brain viability after prolonged untreated cardiac arrest is very high.

In terms of CPR procedures, active compression decompression CPR (ACD CPR) is a type of CPR that has been found beneficial in increasing blood circulation within the body and/or increasing blood oxygenation. ACD CPR increases the amount of blood returned to the heart by enhancing the intrathoracic vacuum or negative pressure during chest wall recoil. The amount of blood circulated within the body during subsequent chest compressions is increased due to the increase in blood that is returned to the heart. ACD CPR is based on the concept that decreasing intrathoracic pressure during the decompression phase of chest compression enhances venous blood return to the thorax, thus “priming the pump” for the subsequent chest compression. In some embodiments, this may be achieved by actively lifting an individual's chest during the decompression phase.

Several devices may be used or assist in performing ACD CPR, such as those described in the patents and applications incorporated herein. For example, a device (e.g., valve system) may be interfaced to a person's airway to enhance circulation. The device/valve system may be used to lower intrathoracic pressure during the chest wall recoil phase of CPR, thereby enhancing the transfer of blood from outside the thorax into the right heart. An Impedance Threshold Device or mechanism (ITD) is one example of a specific device that may be used during performance of CPR or ACD CPR to decrease intrathoracic pressure and improve blood return to the heart. In some embodiments, the ITD device includes a valve that is part of a mask or breathing device (e.g., an endotracheal tube) that opens at a defined high or low pressure. In this way, respiratory gases are prevented from entering the lungs through that valve for at least a portion of each relaxation or decompression phase of CPR. When a threshold negative intrathoracic pressure is achieved, the valve opens and respiratory gases are permitted to flow to the lungs. Several ITD devices are described in the patents and applications incorporated herein. A specific ITD device is the ResQPOD® sold by Advanced Circulatory Systems, Inc.

Another device that may be used is an intrathoracic pressure regulation (ITRP or IPR) device or mechanism. The IPR device may be used to withdraw air from the lungs via an active vacuum source until a negative airway pressure is achieved. In some embodiments, the active vacuum source may be applied intermittently to enhance venous blood flow back to the heart and to decrease intracranial pressures in patients requiring assisted ventilation. This intermittent application of an intrathoracic vacuum may provide a greater hemodynamic benefit to the patient. In some embodiments, a combination of the IPR and ITD devices may be used. For example, an IPR mechanism may include an ITD device or valve, although in such embodiments, the ITD device may function mainly as a safety valve to prevent the generated vacuum from becoming too excessive.

Another form of CPR involves modulating, regulating, or otherwise controlling blood flow to the heart and brain, with or without the administration of a vasodilator drug. This form of CPR involves performing intentional controlled pauses of compressions (i.e., compression “stutter”) during the first 2-3 minutes of the CPR procedure. This form of CPR may provide significant reperfusion injury protection by providing blood to the vital organs in a controlled fashion. This may be particularly useful as changes in blood flow may cause the release of endogenous vasodilators. By modulating blood circulation, potential reperfusion injury following CPR may be reduced. The blood flow may be controlled or modulated so that the vital organs slowly receive additional blood over time. Controlling or modulating blood flow in this manner may involve slowly increasing the amount of blood supplied to the vital organs over time. As described herein, the blood may be circulated using intentional controlled pauses of compressions in a “stutter” fashion where blood is circulated to the vital organs for a certain time, then stopped, then again circulated. This “stutter” CPR approach is more fully described in U.S. Patent Application No. 61/509,994, the complete disclosure of which is herein incorporated by reference.

Preliminary data from a porcine model of prolonged untreated ventricular fibrillation cardiac arrest (i.e., greater than 15 min) strongly suggest that use of intentional controlled pauses of compressions during the first 2-3 minutes provides significant reperfusion injury protection. This approach can be further enhanced by the use of ACD CPR and/or an ITD device. The process can also involve the administration of sodium nitroprusside.

Other forms of cardiopulmonary resuscitation are also possible. Some are performed manually, while others are performed with automated devices. All are designed to improve circulation to the heart and brain. For convenience in describing embodiments of the invention, all forms of cardiopulmonary resuscitation (e.g., ACD CPR, conventional CPR, CPR with ITD devices, CPR with automated devices, etc.) will be referred to herein generally as CPR or CPR procedures. It should be realized that this description does not limit embodiments of the invention to one particular form of cardiopulmonary resuscitation and that all forms of such resuscitating procedures are contemplated by this usage.

In reference to CPR performed during the metabolic phase (i.e., greater than 8 minutes after collapse), there are several fundamental strategies that should be employed together to enhance survival at OHCA. First, blood circulation should be restored rapidly and effectively. Second, the patient should be protected from reperfusion injury. Third, post-resuscitation care of the patient should be optimized. Conventional CPR methods mainly focus on helping to optimize circulation and post resuscitation care without focusing on protecting against reperfusion injury. As such, conventional CPR methods often ignore this under-recognized but critical piece of the enigmatic puzzle, which is a common and harmful contributor to patient morbidity and mortality. For example, the ACD CPR and ITD device techniques described in the above incorporated patents and applications are directed mainly toward optimizing circulation and post resuscitation care.

In contrast, the embodiments described herein focus mainly on reducing reperfusion injury. As such, the embodiments described herein offer potentially significant advancements in CPR effectiveness. When combined with the ACD CPR and ITD device techniques (i.e., the techniques directed to optimizing circulation and post resuscitation care), the embodiments and methods described herein provide a fundamental and relatively complete approach to performing CPR, especially during the metabolic phase.

To protect the heart and brain, and/or other organs, against reperfusion injury, the embodiments described herein are directed toward the idea of delivering an anesthetic to the patient during the performance of CPR. The anesthetic may be delivered to the individual prior to or during reperfusion or reestablishment of blood circulation. For example, an anesthetic may be delivered prior to or during the performance of CPR. In one embodiment, the anesthetic is delivered or introduced during the performance of artificial respiration via an artificial respiration device, such as an ITD or IPR device. For example, an inhaled anesthetic may be delivered immediately upon the start of CPR by professional basic life support (BLS) rescue personnel. In such embodiments, the anesthetic may be delivered with or without an amount of oxygen. The anesthetic may be volatile, or include a volatilizing agent, so that the anesthetic quickly aerosolizes upon breaking of a vial or other container housing the anesthetic. Certain anesthetics (e.g., sevoflurane and the like) have been shown to exert a profound pre-conditioning effect on the brain under other clinical conditions. Studies in support of this concept demonstrate that sevoflurane, when delivered with ACD+ITD CPR (or ACD+IPR CPR), markedly reduces the reperfusion injury associated with prolonged ischemia. The increased circulation afforded by these devices results in a substantially more rapid delivery and distribution of the anesthetic. For example, after administration of a drug (e.g., vasopressin, an anesthetic, and the like) and performance of standard CPR procedures, the drug may accumulate in the peripheral circulation because external chest compressions may not effectively restore systemic circulation. In contrast, the use of enhanced circulation procedures (e.g., ACD CPR and/or the use of an ITD or IPR device) after or during the administration of a drug (e.g., vasopressin, an anesthetic, and the like) may significantly enhance the effectiveness of the administered drug because of improved systemic circulation compared with standard CPR procedures.

In other embodiments, the anesthetic may be delivered to the individual intravenously, intramuscularly, intraosseously, and the like. The anesthetic may be administered or delivered prior to or simultaneously with performing chest compressions. In another embodiment, the anesthetic may be administered prior to restoration of blood circulation or reestablishment of a heartbeat. In some embodiments, the anesthetic is administered through or via an intrathoracic pressure regulation device or mechanism. For example, the anesthetic may be fed through an IPR and/or ITD device, such as the ResQPOD®.

Intravenous drugs may have limited applicability during the early phases of CPR due to their dependence on intravenous access which is generally provided by Advanced Life Support Providers such as medics and physicians. The delay in administration of reperfusion injury protection strategies may result in a loss of benefit when blood flow is introduced more than three minutes before administration of the agent.

This limitation may be circumvented by an approach that involves delivering medications through the lungs using effective methods of CPR that optimize circulation as soon as CPR is initiated. Such methods include ACD CPR, the use of an ITD device with manual CPR, or a combination of ACD CPR and ITD. The ACD CPR+ITD approach is believed to offer the most effectiveness in delivering the medication due to the optimized circulation and post resuscitation care benefits and/or other benefits these procedures provide. In the approaches described herein, access to the lungs is virtually immediate through the airways since access to the lungs can be provided with a bag-valve-mask system or a supraglottic airway device and can be implemented within a few seconds of the resuscitation efforts Inhaled anesthetics such as sevoflurane and isoflurane, helium or xenon can be delivered as a bolus for a short period of time at the first contact with the patients and provide effective protection again ischemia.

Inhaled anesthetics, such as sevoflurane and isoflurane, may provide significant cardiac and cerebral protection from ischemia reperfusion injury when delivered at the initial phase of reintroduction of blood flow in various ischemic models of individual organs. As described herein, such anesthetics may be administered before or during the performance of CPR.

The mechanism through which inhaled anesthetics protect against reperfusion injury is thought to involve protein kinase B and glycogen synthase kinase 3 beta activation as well as protection of the mitochondrial membrane integrity and prevention of cell death, although the exact mechanism of reperfusion injury protection may be multifactorial.

Cardioprotection may be enhanced with administration of an end-tidal concentration of 2-3 vol. % of sevoflurane within the first two minutes of reperfusion. In some embodiments, cardioprotection may be maximal with administration of an end-tidal concentration of 2.4 volume % of sevoflurane for the first two minutes of reperfusion. This short duration of inhaled sevofluorane effectively protects the heart against reperfusion injury in rats in vivo. Longer administration times may offer less cardioprotective effects while accentuating the cardio-depressant effects. Further, sevoflurane may decrease blood and brain oxidative injury and enhance immunity indexes in cerebral ischemia reperfusion. This effect has been observed in rats. According to one theory, it is believed that the protective mechanism could be through inositol trisphosphate kinase/Akt signaling and modulation of Bcl-2 family protein. Administration of an inhaled anesthetic during reperfusion may also offer significant protection to ischemic kidneys. The use of inhaled anesthetics may also offer protection from ischemic reperfusion injury to a variety of other organs.

As described in greater detail below, in one embodiment, a simple mobile anesthesia device can deliver a bolus of inhaled anesthetic of known concentration for the first 3-5 minutes of CPR via an endotracheal tube, facemask or a supraglottic device. In one embodiment, the anesthetic is administered during performance of an enhanced circulation procedure, such as ACD CPR or CPR using an ITD and/or IPR device. The enhanced circulation may deliver the anesthetic to the brain more quickly when compared to conventional CPR methods or other circulation procedures. Enhanced delivery time of the anesthetic to the brain and/or other organs may aid in reperfusion injury protection by allowing the anesthetic to quickly anesthetize the brain and/or other organs. Tissue damage due to reperfusion injury may be reduced when the anesthetic is delivered to the brain quickly after restoration of blood flow or circulation, such as after beginning chest compressions. In one embodiment, the anesthetic may be delivered to the brain within the first several minutes by performing enhanced circulation procedures (e.g., ACD CPR or CPR using an ITD device). In a specific embodiment, the anesthetic is delivered to the brain within the first two minutes or within the first minute after reestablishing blood flow or circulation.

In one embodiment, the anesthetic is administered to the individual in a single dose, although in other embodiments the anesthetic may be delivered via several doses. For example, a single dosage may be administered via intravenous, intramuscular, or intraosseous injection, or may be administered via inhalation. In a specific embodiment, the anesthetic may be volatilized and delivered via one or more respirations. In inhalation administration procedures, the anesthetic may be delivered for 30 seconds, 2 minutes, and more commonly for 5 minutes or more. In another embodiment, a dosage of the anesthetic may be delivered during each respiration or during repeated respirations for a portion of the time an individual receives artificial respiration, or the entire period of time the individual receives artificial respiration. In such embodiments, delivery of the anesthetic may last for 30 minutes or more, often at reduced dosage amounts, in order to provide effective post resuscitation care. In one embodiment, the anesthetic (e.g., sevoflurance) may be delivered in a dosage amount of 2-4 vol % over one or more respirations. In another embodiment, the anesthetic may be delivered during the short and intentional periodic pauses in a “stutter” CPR process.

In one embodiment, the administered anesthetic may include sevoflurane. In other embodiments, various other volatile anesthetics may be administered including: halothane, enflurane, desflurane, isoflurane, xenon, helium, and the like. Sevoflurane may provide a significant advantage over other inhaled anesthetics by providing significant protection from reperfusion injury. The use of sevoflurane may also result in less cardio-depressant effects.

These anesthetics may offer protective effects when administered before or during CPR and help stabilize the brain, heart, or body to help the heart, brain, and/or body stay relatively fresh. It is believed that some anesthetic agents provide these effects by attenuating the mitochondrial membrane permeability, or manipulating and protecting the mitochondrial membrane pores. For example, during ischemia, permeability pores in the mitochondria cell membrane open up causing the mitochondria to swell. In such a condition, the mitochondria may be destroyed or damaged when blood flow is reestablished. The anesthetic may help protects against such damage, especially if the anesthetic is delivered or delivery is initiated early in reestablishing blood flow. Some volatile anesthetics like Xenon provide anesthesia by non-competitively inhibiting N-methyl-D-aspartate receptors.

Due to the fact that reperfusion injury occurs very early during the resuscitation efforts (e.g., within 2-3 minutes from the imitation of CPR), it is preferable that the anesthetic be delivered, or that delivery be initiated, within the first 5 minutes of beginning CPR efforts, and preferably within a minute or less of beginning CPR. In other embodiments, the anesthetic may be delivered, or delivery initiated, within the first 2-3 minutes of beginning CPR, or prior to the performance of CPR. This early delivery of the anesthetic may provide enhanced protection by reducing or preventing the initial shock the organs experience by the first pass of blood and oxygen. This early delivery may also or alternatively prevent or reduce the effects of adverse toxins that may accumulate in the blood during ischemia. Early delivery of the anesthetic may further aid in resuscitating the individual. For example, in animal studies involving administration of an anesthetic gas during CPR, the animal was resuscitated after only one or two shocks. Furthermore, within an hour or two of resuscitation, most or all the animals' functions were normal even without administration of other drugs, such as inotropic drugs.

It is believed that the anesthetic may also help modulate the autonomic nervous system by altering the body's response to adrenaline and/or the nervous system's response to restarting blood flow or circulation. Subsequent to establishing blood circulation and/or reestablishing a heartbeat, the body experiences a surge of adrenaline and other stress hormones. The anesthetic may modulates the body's response and/or blunt the nervous system's response to the adrenaline and/or other stress hormones levels associated with reestablished circulation.

The anesthetic may further provide protection by allowing or enabling energy stores of neurons to be replenished before they can be activated again. This may help to reduce the mismatch between energy supplies and energy stores in the brain and/or other areas of the body. For example, the anesthetic may reduce the energy need of the brain, thereby ensuring that the brain does not quickly exhaust its fuel supply and extending the functional time of the brain. Delivering and circulating the anesthesia to the brain early in the CPR process may metabolically stabilize the brain cells and preserve them so that they remain subdued. The anesthetic may similarly reduce the activity of the central nervous system and thereby reduce its need for fuel. Accordingly, the anesthetic may prevent or prolong these systems from functioning in an energy deprived state or condition.

In one embodiment, the anesthetic is delivered prior to establishment of a heartbeat or blood circulation, artificial or otherwise, such as via chest compressions, defibrillation, and the like. More commonly, the anesthetic is delivered simultaneously with or quickly after establishing a heartbeat or blood circulation, by artificial support or otherwise. Preferably, the anesthetic is delivered within the first 5 minutes of re-establishing a heartbeat or blood circulation, within the first 3 minutes, or within the first 2 minutes.

In animal studies where an anesthetic gas was administered as described herein, heart muscle damage was drastically reduced. In many of the animals that received the anesthetic, the heart was beating normally and blood pressure was normal after approximately one hour of resuscitation. The ability to resuscitate the animal was also enhanced. These and other aspects of the invention will become more evident in light of the description of figures provided below.

Administration Devices

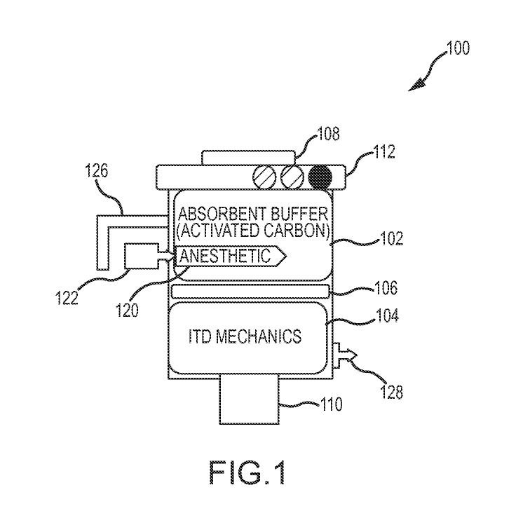

FIG. 1 illustrates a device 100 or system for administering an anesthetic to an individual to reduce reperfusion injury while performing CPR. Device 100 may be used to deliver medication, such as the described anesthetics, via the patient's airway to enable the medication to quickly and easily access to the patient's lungs. In some embodiments, device 100 may employ a bag-valve-mask system device that can be implemented and fit about the patient within a few seconds of the resuscitation efforts. The medication (e.g., inhaled anesthetic sevoflurane) can be delivered as a bolus for a short period of time at the first contact with the patients to provide effective reperfusion injury protection.

In some embodiments, device 100 is a disposable, lightweight device that includes an ITD and a unit-dose, vaporized anesthetic delivery and scavenger mechanism. The device 100 can be used for performing CPR with the ITD technology as well as being able to safely provide reperfusion injury protection with sevoflurane or another anesthetic. Device 100 may be used to introduce a vaporized halogenated inhalation anesthetic into the breathing system of a patient receiving CPR. Device 100 may also largely absorb the patient's exhaled anesthetic vapor to protect caregivers from anesthetic exposure. Device 100 may include a unit-dose liquid injection means to provide the specified amount of anesthetic vapor for a specified length of time. Use of an ITD or IPR device is preferred as these devices have been shown to enhance circulation and thus drug delivery during CPR. For example, as described herein, the increased circulation afforded by these devices results in a substantially more rapid delivery and distribution of the anesthetic. The use of enhanced circulation procedures (e.g., ACD CPR and/or the use of an ITD or IPR device) after or during the administration of a drug (e.g., vasopressin, an anesthetic, and the like) may significantly enhance the effectiveness of the administered drug because of improved systemic circulation compared with standard CPR procedures.

As shown in FIG. 1, device 100 includes a housing having an inlet or ventilation port 108 and an outlet or patient port 110. Inlet port 108 is configured to connect with a ventilation source or device that is used to provide oxygen or other gas to the individual. In some embodiments, the ventilation source may include a compressible bag, a mechanical device, an anesthesia machine, a ventilator, and the like. Inlet port 108 may be pivotally coupled with the housing to allow the ventilation source or device to be attached and used within a wide range of angles. The housing may also include various electronics or guidance systems 112 that guide a user or operator in performing CPR. For example, guidance system 112 may guide a user on proper ventilation rate and respiration duration and/or may indicate or signal a timing of when to provide subsequent respirations. One or more lights may be used that indicate: 1) when an artificial breath or respiration should be provided, 2) the duration of the respirations that should be administered, 3) when chest compressions should be performed, and the like. In another embodiment audio signals can be used to provide similar user feedback and instructions. In one embodiment, the lights are configured to indicate respirations and chest compressions according to the “stutter” CPR process so that CPR process involves intentional short periodic pauses as described herein. The anesthetic may be administered during one or more of these short periodic pauses and/or at any other time during the process. Outlet port 110 typically connects with tubing and/or a mask that is placed over the patient to deliver oxygen and/or inhalants to the patient, such as the anesthetic.

Device 100 may also include an upper chamber 102 and a lower chamber 104 separated by a filter material or membrane 106. Upper chamber 102 may include a port 120 or chamber for receiving the anesthetic. In one embodiment, an ampule or vial of the anesthetic may be inserted within port 102. A fracture button 122 may then be pressed to break the ampule or vial and thereby release the anesthetic within upper chamber 102. In some embodiments, fracture button 122 may also be used to inject the anesthetic within upper chamber 102. As described herein, the anesthetic may include a volatile agent that allows the anesthetic to quickly aerosolize. Upper chamber 102 may include an absorbent buffer, such as activated carbon, which is used as a capture chamber to scavenge the gas and keep the anesthetic from escaping. Using the scavenging system, the gas could be recirculated. In one embodiment, the activated carbon includes charcoal.

The vial or ampule inserted or injected within port 102 may include a single dose of the anesthetic. In one embodiment, the single dose may provide approximately 30 seconds worth of inhalant anesthetic. In another embodiment, the single dose may provide approximately 5 minutes or more worth of inhalant anesthetic. In yet another embodiment, the single dose may provide between about 30 seconds and 5 minutes worth of inhalant anesthetic. In some embodiments, multiple vials/doses may be administered to the patient depending on the volume of anesthetic gas, respiration duration, and/or frequency of respirations desired. In one embodiment, approximately a half a liter of anesthetic gas may be provided in a single respiration, which may be administered for about 30 seconds. In another embodiment, a series of about 5 respirations may be provided where each respiration contains about half a liter (i.e., approximately 500-525 cubic centimeters) of anesthetic gas so that a total volume of approximately 2500 cubic centimeters of anesthetic gas is administered. As described herein, administration of the anesthetic gas may be initiated within the first 5 minutes of performing CPR, and preferably within the first 2 or 3 minutes or prior to initiation of CPR.

In a specific embodiment, the anesthetic may be administered within the first 2-3 minutes or less of starting CPR and may be administered for at least 30 seconds and more commonly about 5 minutes or more. In some embodiments, the anesthetic may be delivered during the intentional short periodic pauses in a “stutter” CPR process. The above described volumes and durations may be sufficient to ensure that the brain and/or other organs are sufficiently anesthetized early in CPR process, which may effectively protect against reperfusion injury by reducing or eliminating damage to the body's organs/tissue as described herein. Delivering the anesthetic gas early in the reperfusion process may provide additional protection against reperfusion injury since it is believed that reperfusion injury often occurs or begins within the first 3 minutes of reestablishing blood flow.

In addition to administering the anesthesia early in the process, in one embodiment the anesthetic gas may be delivered during a majority the CPR process or during the entire CPR process. In some embodiments, the anesthetic may be delivered for up to 24 hours while the patient is recovering.

In one embodiment, a liquid anesthetic is injected into upper chamber 102 and subsequently aerosolized via an aerosolizer. Also, although shown as being positioned in the upper chamber 102, in some embodiments, the vial or ampule chamber 120 may be positioned in the lower chamber 104.

Filter 106 is used to filter gas that passes from upper chamber 102 through lower chamber 104 and to the patient or individual. Specifically, filter 106 is used to remove bacteria and other microscopic particles that could infect or otherwise harm the patient or individual. In one embodiment, filter 106 removes bacteria as small as 10 microns. The gas from inlet port 108 and aerosolized anesthetic gas passes through filter 106 into lower chamber 104, which is positioned below filter 106. In one embodiment, the gas from inlet port 108 is oxygen that is mixed with the anesthetic in upper chamber 102. Lower chamber 104 includes an intrathoracic pressure regulation mechanism or device (e.g., and ITD and/or IPR device) that prevents or impedes respiratory gases from flowing to the lungs, or that otherwise generates a vacuum or regulates flow through or pressure within the patient's airway. In one embodiment, lower chamber 104 includes an impedance threshold device (ITD), such as an inspiratory limb flow control assembly or inspiratory valve mechanism. As described above, the ITD or IPR device functions to increase blood return to the heart thereby improving blood flow during subsequent chest compressions. Exemplary ITD devices that may be used with device 100 include those described in U.S. Pat. Nos. 5,551,420; 5,692,498; 5,730,122; 6,062,219; 6,155,257; 6,224,562; 6,234,916; 6,526,973; 6,604,523; 6,776,156; 6,986,349; 7,195,012; and 7,204,251; and U.S. Provisional Patent Application No. 61/577,565, the complete disclosures of which are herein incorporated by reference. In some embodiments, device 100 may include a safety cover 126 that is positioned over fracture button 122 and/or port 120. Device 100 may also include a supplemental oxygen/gas port 128 that is fluidly coupled with lower chamber 104.

In some embodiments, device 100 may be optimized to keep rebreathed or recirculated air deadspace to a minimum. For example, a flow of 0.5 to 1 liters/minute (lpm) of respiratory gas that is introduced between the patient and the device may partially flush the deadspace in the media. To compensate for deadspace, encapsulated absorbant such as soda lime may be included with the adsorption media to absorb CO2. This absorption may produce a small amount of heat and moisture for added humidification and warming of the patient's inspired gas.

Inhalation agents, in liquid form, typically easily vaporize in air. The introduction of small amounts of liquid agent to breathing gas may result in assured vaporization of the liquid, though care should be taken to ensure that the liquid never contacts the patient's airway. The delivery of the inhalation agent may be buffered by the device to prolong and flatten the peak vapor concentration over the first few minutes of CPR. Exhaled vapor may be adsorbed and re-introduced to inhalation gas. Ventilation may be supplied by a self-refilling manual resuscitator or a transport ventilator. The buffering effect may be created by a buffering medium which slows the release of anesthetic agent into inspired gas. The media may also provide an adsorption effect, where exhaled agent is adsorbed during exhalation and released during inspiration.

As it is released from the device, exhalation gas typically passes through the absorption media (activated charcoal), which absorbs the majority of the anesthetic vapor as it passes from the exhalation port of device 100. The pharmacokinetics of the inhalation agent uptake may be defined by the equation:

$$Uptake = \frac{Partition Coefficient * Alveolar}{Venous difference * Cardiac Output}$$

In the above equation, the sevoflurane blood gas partition coefficient at 37° C. is typically 0.65. For each 1 ml of sevoflurane liquid, 182 ml of vapor may be produced. The vapor may be taken up by the patient according to the agent's partition coefficient (above equation), but in general, the patient will consume the delivered vapor at a rate inversely proportional to the square root of time. In some embodiments, the predicted efficiency of the adsorption media may be estimated to be greater than 80%; meaning that 80 to 90 percent of the exhaled agent will be re-inspired. The entire system may be tuned using the deterministic formula above and then tested to ensure that the predicted performance is achieved in the physical properties and design of the device 100.

The majority of sevoflurane may be eliminated from the body through breathing. Since sevoflurane will be eliminated through exhalation, the patient will normally shed the halogenated agent when the device is removed post ROSC. As such, the CPR patient may recover similar to an anesthesia patient recovering from intraoperative levels of anesthetic, though the quantum volume of anesthetic required for reperfusion injury protection is commonly less than the amount required for surgery.

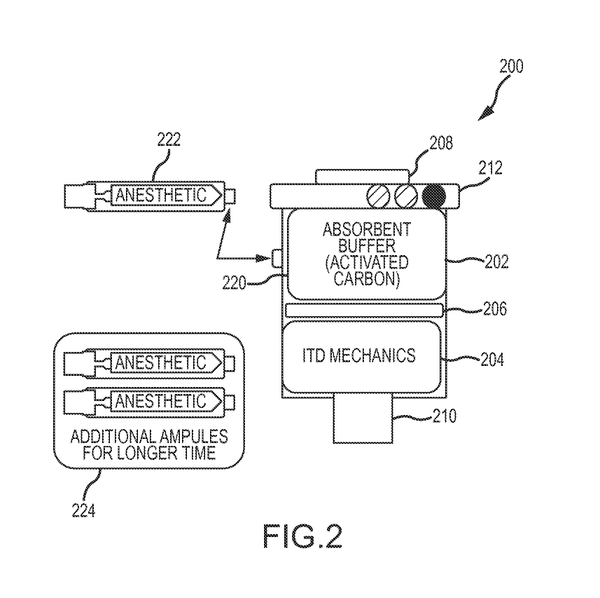

FIG. 2 illustrates another embodiment of a device 200 or system for administering an anesthetic to an individual to reduce reperfusion injury. Device 200 is similar to device 100 in that it includes a patient port 210, a ventilation port 208, various electronics or guidance systems 212, an upper chamber 202, a lower chamber 204, a filter 206 separating the upper chamber and lower chamber, and an anesthetic port 220 within which a liquid anesthetic 222 may be injected. Anesthetic port 220 may an aperture or plug that extends from the housing and that couples with a distal end of the vial or ampule containing the liquid anesthetic 222. The vial or ampule containing the liquid anesthetic 222 may be broken or cracked open and injected into upper chamber 202 and/or lower chamber 204. In some embodiments, the liquid anesthetic may be volatile or include a volatilizing agent so that the liquid anesthetic instantly transitions to a gas upon injection. In other embodiments, upper chamber 202 may include an aerosolizer that aerosolizes the liquid anesthesia. According to one embodiment, the vial or ampule of liquid anesthesia may be administered to the patient over a 30 second interval, 2 minute interval, 3 minute interval, 5 minute interval, and the like. According to another embodiment, additional vials or ampules 224 of the anesthesia may be used depending on the amount of anesthesia to be administered and/or depending on the duration of administration of the anesthesia.

According to one embodiment, a method of using the device 100 or 200 includes a rescuer or user of the device 100 or 200 arriving at a location where CPR is needed. The rescuer opens a vial or ampule of the anesthetic liquid or gas and inserts or injects the anesthetic liquid or gas into device 100 or 200 via respective chambers 120 or 220. The rescuer then begins to ventilate the individual needing CPR with the anesthetic gas, plus/minus any oxygen as desired. Ventilation occurs for 30 seconds, 2 minutes, 3 minutes, 5 minutes, or more depending on the individual's need and or other factors. Additional vials or ampules may be needed to provide the desired ventilation. The rescuer then performs, or simultaneously performs, chest compressions or defibrillation to establish blood circulation within the individual's body. The individual could then be ventilated with or without the anesthetic gas.

Methods

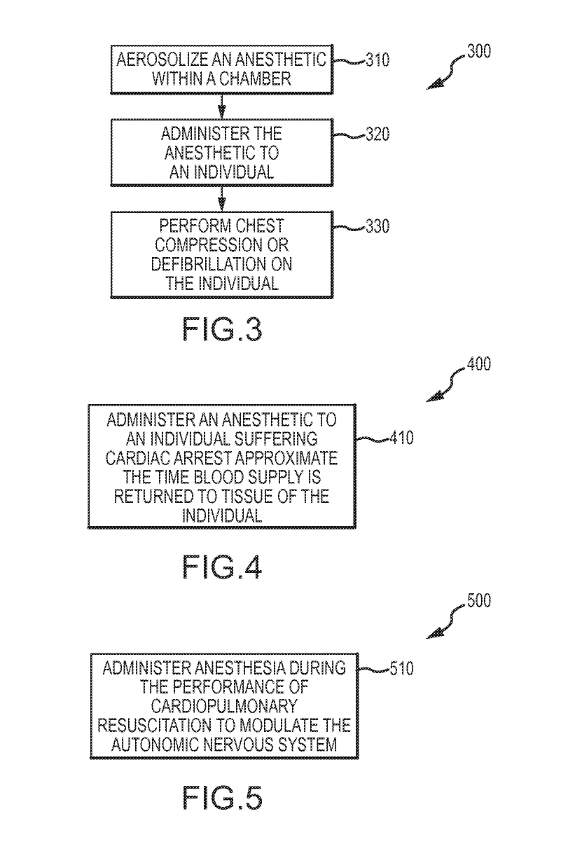

FIG. 3 illustrates a method 300 of performing cardiopulmonary resuscitation on an individual while administering an anesthetic to the individual. Method 300 may be performed while using a CPR device, such as device 100 or 200. At block 310, an anesthetic is aerosolized within a chamber, such as by injecting anesthesia into the chamber from a vial or ampule. The anesthesia may aerosolize on its own or be aerosolized with the use of a device. As described above, in some embodiments the anesthetic is administered intravenously, intramuscularly, intraosseously, and the like. In such embodiment, blocks 310 and 320 are not performed. At block 320, the anesthesia is administered to the individual, such as by providing artificial respiration to the individual via a ventilator, a ventilation bag, and the like. Artificial respiration may be provided to the individual in accordance with known CPR techniques or procedures, such as ACD CPR. In one embodiment, the anesthetic is administered to the individual for at least 30 seconds. In another embodiment, the anesthetic is administered to the individual for at least 5 minutes. As described above, the anesthetic may be administered for more or less time than this depending on the individual's needs and/or other circumstances. The CPR procedure may utilize an IPR and/or ITD device through which the anesthetic may flow.

According to one embodiment, the anesthesia may be mixed and administered with oxygen. In another embodiment, a series of anesthesia treatments may be administered with gaps between subsequent treatments. For example, artificial respiration may be provided at repeated intervals after chest compressions or defibrillation is administered according to known guidelines or procedures (e.g., 2 breaths for every 30 compressions). In some embodiments, the anesthesia may be administered during an initial or first set of artificial respirations, but not administered during the next or second set of artificial respirations. The anesthesia may then again be administered during a third set of artificial respirations while skipping administration during a fourth set of artificial respirations. This cyclic administration process may continue for as long as artificial respiration is needed.

In another embodiment, the anesthesia may be administered every tenth artificial respiration so that a gap of approximately nine respirations occurs between subsequent anesthesia administrations. It should be realized that other anesthesia administration/gap combinations are possible depending on the patient's need, the patient's condition, and/or any other condition.

In some embodiments, oxygen is administered during artificial respiration when the anesthesia is not administered. In another embodiment, oxygen is administered during each or most of the artificial respirations and, when administered, the anesthesia is mixed and administered with the oxygen. In another embodiment, the oxygen or gas administered may be recirculated to the individual.

At block 330, chest compressions or defibrillation are provided to the individual in accordance with known CPR techniques or procedures. In one embodiment, the anesthetic is administered to the individual prior to performing any chest compressions or defibrillation so that anesthesia is delivered to the body, or portions thereof, prior to or simultaneously with reestablishing blood circulation. In another embodiment, the anesthetic is administered to the individual simultaneously with performing chest compressions or defibrillation, or after one or more chest compressions or defibrillation procedures have been performed.

In either embodiment, the anesthetic may be administered early in the CPR process and/or prior to restoration of a heartbeat. According to another embodiment, the anesthetic is administered while performing enhanced circulation cardiopulmonary resuscitation, such as ACD CPR or CPR using an ITD/IPR device. As described above, the anesthetic may modulate the autonomic nervous system of the individual.

FIG. 4 illustrates a method 400 of reducing reperfusion injury after a period of cardiac arrest and no blood flow. At block 410, an anesthetic is administered to an individual suffering ischemia prior to or approximate the time blood supply is returned to tissue of the individual. The anesthetic may be administered during the administration of cardiopulmonary resuscitation to the individual.

FIG. 5 illustrates another method 500. At block 510, anesthesia is administered during the performance of cardiopulmonary resuscitation to modulate the autonomic nervous system.

The embodiments described herein provide multiple examples of methods and devices that can be used to provide reperfusion injury protection during the first few minutes of CPR. It should be noted that other variations are also possible and thus these examples are not meant to be limiting. In this regard, it is also contemplated to deliver several reperfusion protection agents simultaneously, which may provide synergistic effects. For example, sevoflurane could be delivered along with Cyclosporine A, which is a drug known to block a key mitochondrial permeability pore. The combination of these two drugs, delivered simultaneously through an aerosolized preparation into the lungs, may be synergistic as different mechanisms of protection are deployed, and in this manner could provide further protection of the heart and brain against reperfusion injury.

### Example

As an example, Applicants conducted experiments on pigs experiencing induced cardiac arrest. Cardiac arrest was induced by generating ventricular fibrillation (VF) and pigs were left in untreated cardiac arrest for 20 minutes. At the initiation of conventional manual CPR, inhaled isoflurane at 4% was introduced via an anesthesia machine for a total of 3 minutes. The anesthesia was then turned off. After a total of 4 minutes of CPR the animals were then shocked back into a stable perfusing heart rhythm and the dose of isoflurane anesthesia was restarted at 1% to provide for anesthesia during the recovery period of the animals. In all 4 animal studies, return of spontaneous circulation (ROSC) was achieved with 1-2 shocks after 4 minutes of CPR and one dose of 0.5 mg of epinephrine. Cardiac function based upon an assessment of the left ventricular (LV) ejection fraction (EF) after 1 and 4 hours was normal, as determined by echocardiography, and post resuscitation inotropic support was unnecessary due to the absence of hemodynamic instability. Two of the 4 animals had undetectable levels of markers of cardiac injury (creatinine phosphokinase MB and cardiac troponin [cTnI]), while 2 of the 4 animals had mild elevations of markers of cardiac injury (creatinine phosphokinase MB and cTnI) at 4 hours post ROSC (6.8±9 and 5.8±11 in ng/mL, respectively).

These results represent a significant improvement compared to control animals treated with conventional manual CPR after 15 minutes of untreated VF and no anesthetic. Although the duration of untreated VF in the control animals was 5 minutes shorter, post ROSC left ventricular function was severely compromised (EF: 33±9%) and there was significant elevation of CKMB and cTnI at 4 hours post ROSC (26.5±13.7 and 31.2±14.3, p<0.05) compared to isoflurane treated animals resuscitated after 20 minutes of untreated VF. In the absence of anesthesia during CPR as described above, Applicants were been unable to resuscitate pigs after 20 minutes of untreated arrest with conventional CPR. These findings represent an important advance in the field of CPR.

Another experiment was conducted with similar results. In that experiment, the hearts were subsequently removed from 5 animals from each group 15 minutes post-ROSC for mitochondrial function testing. Compared with the control group, all mitochondrial function tests were improved in the sevoflurane treated animals. These results of the experiment are presented in Table 1 below.

TABLE 1

| | Controls | SPoC |
|---|---|---|
| LVEF (%) 12 min | 37 ± 12 | 53 ± 16* |
| Post ROSC | | |

Mitochondrial Function

| | | |
|---|---|---|
| RCI P/M | 7.1 ± 0.9 | 11.4 ± 0.5* |
| RCI Suc | 2.9 ± 0.4 | 4.5 ± 0.2* |
| ATP P/M | 75.5 ± 13.3 | 148.5 ± 24.8* |
| [nm/min/mg] | | |
| ATP Suc | 73.2 ± 11.3 | 132.3 ± 18.4* |
| [nm/min/mg] | | |
| Ca Ret P/M | 178.4 ± 32.4 | 318.8 ± 45.0* |
| [nm/min/mg] | | |
| Ca Ret Suc | 228.2 ± 57.0 | 474.0 ± 58.8* |
| [nm/min/mg] | | |

Table 1 illustrates the mitochondrial function for complex I substrates pyruvate (P, 10 mM) and malate (M, 10 mM) and complex II substrate succinate (Suc, 10 mM) in control hearts vs. sevoflurane-treated hearts (SPoC). LVEF refers to the left ventricle ejection fraction percentage at 12 min post return of spontaneous circulation (ROSC). RCI refers to the respiratory control index (state 3/state 4 respiration). ATP refers to adenosine triphosphate synthesis and Ca Ret refers to calcium retention capacity until mitochondrial transition pore opening. The asterisk (*) refers to P of less than 0.05.

What is claimed is:

1. A device for delivering an anesthetic to an individual to reduce reperfusion injury during the performance of cardiopulmonary resuscitation (CPR), the device comprising:
a patient connection mechanism for coupling with an airway of the individual;
a pressure regulation device that is configured to regulate a negative intrathoracic pressure of the individual while CPR is being performed on the individual; and
an anesthetic delivery mechanism for receiving the anesthetic and for delivering the anesthetic to the individual via the patient connection mechanism.

2. The device of claim 1, wherein the device comprises a housing, and wherein the anesthetic delivery mechanism comprises a chamber for receiving a vial of the anesthetic.

3. The device of claim 1, wherein the pressure regulation device comprises an intrathoracic pressure regulation (IPR) mechanism coupled with the patient connection mechanism, the IPR mechanism being configured to change a pressure in the airway and a thorax of the individual via the application of a vacuum source.

4. The device of claim 1, wherein the device comprises a housing having an upper chamber and a lower chamber separated by a filter, wherein the lower chamber comprises an impedance threshold device (ITD), and wherein the upper chamber comprises an inlet port for receiving a gas to be delivered to the individual via the patient connection mechanism.

5. The device of claim 4, wherein the upper chamber further comprises an absorbent buffer.

6. The device of claim 1, wherein the device further comprises an aerosolizer to aerosolize the anesthetic.

7. A method of performing cardiopulmonary resuscitation comprising:
repeatedly compressing an individual's chest, wherein the chest is compressed during a compression phase followed by a decompression or relaxation phase; and
administering an anesthetic to the individual receiving cardiopulmonary resuscitation.

8. The method of claim 7, wherein the anesthetic is administered through the lungs of the individual within 3 minutes of initiating cardiopulmonary resuscitation.

9. The method of claim 8, wherein the anesthetic is administered through the lungs of the individual within 30 seconds of initiating cardiopulmonary resuscitation.

10. The method of claim 7, wherein the anesthetic is administered prior to compressing the individual's chest or applying a defibrillating shock.

11. The method of claim 7, wherein the anesthetic is administered while performing an enhanced circulation cardiopulmonary resuscitation procedure including:
a procedure involving an impedance threshold device (ITD) where respiratory gases are prevented from entering the lungs for at least some time during the decompression or relaxation phase;
manual or automated active compression decompression (ACD) CPR where the individual's chest is actively lifted during the decompression phase;
a procedure involving an intrathoracic pressure regulator (IPR) device where the IPR mechanism changes a pressure in the airway and a thorax of the individual via application of a vacuum source; or
a combination thereof.

12. The method of claim 7, wherein the anesthetic is administered prior to restoration of a heartbeat.

13. The method of claim 7, wherein the anesthetic modulates the autonomic nervous system.

14. The method of claim 7, wherein the anesthetic comprises one or more selected from the group consisting of:
isoflurane;
sevoflurance;
xenon;
helium;
desflurane;
enflurance;
halothane;
methoxyflurance;
nitrous oxide;
propofol;
ketamine;
etomidate;
amobarbital;
methohexital;
thiopental;
a barbiturate; and
a benzodiazepine.

15. The method of claim 14, wherein two or more doses of the anesthetic or two or more anesthetics are administered to the individual receiving cardiopulmonary resuscitation.

16. The method of claim 15, wherein the two or more anesthetics are administered substantially simultaneously.

17. The method of claim 7, wherein the anesthetic is administered to the individual via inhalation or intravenously.

18. The method of claim 17, wherein the anesthetic is administered to the individual for at least 30 seconds.

19. The method of claim 18, wherein the anesthetic is administered to the individual for at least 5 minutes.

20. A method of reducing reperfusion injury after a period of ischemia, the method comprising:
administering an anesthetic to an individual prior to or approximate the time blood supply is returned to tissue of the individual.

21. The method of claim 20, wherein the anesthetic is administered during cardiopulmonary resuscitation.

22. A method comprising:
performing cardiopulmonary resuscitation to an individual by repeatedly compressing an individual's chest, wherein the chest is compressed during a compression phase followed by a decompression or relaxation phase;
administering anesthesia during the performance of cardiopulmonary resuscitation to modulate the autonomic nervous system of the individual.
